(19) United States
(12) Patent Application Publication
(10) Pub. No.: US 2018/0014817 A1
(43) Pub. Date: Jan. 18, 2018

(54) Biological Specimen Collection Apparatus

(71) Applicant: BETTA, LLC, Orem, UT (US)

(72) Inventors: Ricardo Cugler Janotti, Pleasant Grove, UT (US); Valeria Felix Barbour, Provo, UT (US)

(21) Appl. No.: 15/641,506

(22) Filed: Jul. 5, 2017

Related U.S. Application Data
(60) Provisional application No. 62/363,659, filed on Jul. 18, 2016.

(51) Int. Cl. A61B 10/00; A61F 5/451; A47K 11/10

(52) U.S. Cl. CPC A61B 10/0038; A47K 11/105; A61B 10/007; A61F 5/451; A61B 2090/0807; A47K 2201/00

(57) ABSTRACT

A biological specimen collection apparatus may include markings for facilitating and positioning a sheet of material relative to an opening of a toilet seat so as to assist a user to properly depositing a biological specimen in the apparatus. The apparatus may also include a plurality of volumetric markings.

### BACKGROUND

### 1. Technical Field

The present invention relates to a device for obtaining biological samples or specimens. More specifically, the present invention relates to a device which mounts on a toilet so as to obtain urine, stool, vomit, or other biological samples from a patient.

### 2. State of the Art

There are many occasions in which a medical professional needs to obtain a biological sample or specimen from a patient. Stool samples are often used to detect various cancers, pathogens, and other diseases. Vomit samples are often collected to isolate pathogens which may be causing the vomiting. Likewise, urine samples are often obtained to determine malfunctions in various organs or to look for other indications of disease. In the past there have been a variety of devices for collecting such specimens, for example, a plastic bag is placed over a toilet seat to collect a stool sample, the plastic bag, however, includes a plurality of holes which allow urine to escape.

Other devices have been made which mount on a toilet seat which have a solid bottom but can be difficult to place on or remove from the toilet seat. A person attempting to remove the device may inadvertently spill the contents in the process. Additionally, there are some situations in which it's important to have an approximation of the volume of the vomit, urine sample, etc. as a factor of biological significance.

While there are devices which handle various collection issues relatively well, each has its own drawbacks with respect to collecting biological samples. The present application discloses a new biological collection system which resolves many of these concerns.

### SUMMARY OF THE INVENTION

The present invention provides a biological specimen collection apparatus which includes a flexible piece of material which may be disposed between a toilet seat and a toilet bowl so as to catch biological samples as they are excreted or otherwise released from the patient.

In accordance with one aspect of the invention, the piece of material has a first, lower layer or sheet which is disposable between the toilet seat and the bowl, and a second, upper layer or sheet which is disposed above the toilet seat. The first, lower layer is substantially solid and may include thereon markings to give an approximation of the volume collected therein. The second, upper layer, in contrast, has an opening which is sized to align with a toilet seat so that the second layer sits above the toilet seat and the user sits thereon. A hole in the upper layer aligns with a hole in the toilet seat to allow specimens to pass to the lower layer.

At one end the sheets are unattached so as to enable a toilet seat to slide between the first, lower layer and the second, upper layer. The sides and front end are generally sealed or formed from a single piece of material so as to limit movement rearwardly and side to side as the device is placed on the toilet seat.

In accordance with one aspect the invention, the forward edge of the device may be rounded so as to follow the general shape of a toilet seat. In another variation, the overall construction is rectangular.

In accordance with another aspect of the invention, the second, upper layer may include a marker to facilitate alignment of the second sheet above the opening in a toilet seat.

In accordance with another aspect of the invention the sample collection device may be made with a single sheet of material. The single sheet of material may include a plurality of adhesive members disposed thereon such that the single sheet of material can be adhered to a toilet bowl to prevent the sheet from moving. The sheet may also include markings to facilitate alignment of the sheet relative to a toilet seat and may also include markings to estimate volume of biological sample received in the device.

### BRIEF DESCRIPTION OF THE DRAWINGS

Various embodiments of the present invention are shown and described in reference to the numbered drawings wherein:

FIG. 1 shows a perspective view of a biological specimen collection apparatus in accordance with one aspect of the present disclosure;

FIG. 2 shows a top view of the biological specimen collection apparatus of FIG. 1;

FIG. 3 shows a bottom view of the biological specimen collection apparatus of FIG. 1;

FIGS. 4 and 5 show side views of the biological specimen collection apparatus of FIG. 1;

FIGS. 6 and 7 show end views of the biological specimen collection apparatus of FIG. 1;

FIG. 8 shows a perspective view of an alternate embodiment of the biological specimen collection apparatus;

FIGS. 9 and 10 show top and bottom views, respectively, of the biological specimen collection apparatus of FIG. 8;

FIGS. 11 and 12 show sides views of the biological specimen collection apparatus of FIG. 8;

FIGS. 13 and 14 show end views of the of the biological specimen collection apparatus of FIG. 8;

FIG. 15 shows an alternate embodiment of a biological specimen collection apparatus;

FIGS. 16 and 17 show top and bottom views, respectively, of a biological specimen collection apparatus of FIG. 15;

FIGS. 18 and 19 show side views of the biological specimen collection apparatus of FIG. 15; and

FIGS. 20 and 21 show end views of the biological specimen collection apparatus of FIG. 15.

It will be appreciated that the drawings are illustrative and not limiting of the scope of the invention which is defined by the appended claims. The embodiments shown accomplish various aspects and objects of the invention. It is appreciated that it is not possible to clearly show each element and aspect of the invention in a single figure, and as such, multiple figures are presented to separately illustrate the various details of the invention in greater clarity. Similarly, not every embodiment need accomplish all advantages of the present invention.

### DETAILED DESCRIPTION

The invention and accompanying drawings will now be discussed in reference to the numerals provided therein so as to enable one skilled in the art to practice the present invention. The skilled artisan will understand, however, that the apparatuses, systems and methods described below can be practiced without employing these specific details, or that they can be used for purposes other than those specifically described herein. Indeed, they can be modified and can be used in conjunction with products and techniques known to those of skill in the art in light of the present disclosure. The drawings and descriptions are intended to be exemplary of various aspects of the invention and are not intended to narrow the scope of the appended claims. Furthermore, it will be appreciated that the drawings may show aspects of the invention in isolation and the elements in one figure may be used in conjunction with elements shown in other figures.

Reference in the specification to “one embodiment” or “an embodiment” means that a particular feature, structure, or characteristic described in connection with the embodiment is included in at least one embodiment, but is not a requirement that such feature, structure or characteristic be present in any particular embodiment unless expressly set forth in the claims as being present. The appearances of the phrase “in one embodiment” in various places may not necessarily limit the inclusion of a particular element of the invention to a single embodiment, rather the element may be included in other or all embodiments discussed herein.

Furthermore, the described features, structures, or characteristics of embodiments of the invention may be combined in any suitable manner in one or more embodiments. In the following description, numerous specific details are provided, such as examples of products or manufacturing techniques that may be used, to provide a thorough understanding of embodiments of the invention. One skilled in the relevant art will recognize, however, that embodiments of the invention may be practiced without one or more of the specific details, or with other methods, components, materials, and so forth. In other instances, well-known structures, materials, or operations are not shown or described in detail to avoid obscuring aspects of the invention.

Before the present invention is disclosed and described in detail, it should be understood that the present disclosure is not limited to any particular structures, process steps, or materials discussed or disclosed herein, but is extended to include equivalents thereof as would be recognized by those of ordinarily skill in the relevant art. More specifically, the invention is defined by the terms set forth in the claims. It should also be understood that terminology contained herein is used for the purpose of describing particular aspects of the invention only and is not intended to limit the invention to the aspects or embodiments shown unless expressly indicated as such. Likewise, the discussion of any particular aspect of the invention is not to be understood as a requirement that such aspect is be present apart from an express inclusion of the aspect in the claims.

It should also be noted that, as used in this specification and the appended claims, singular forms such as “a,” “an,” and “the” may include the plural unless the context clearly dictates otherwise. Thus, for example, reference to “a spring” or “a pack” may include one or more of such springs or packs, and reference to “the layer” may include reference to one or more of such layers.

As used herein, the term “substantially” refers to the complete or nearly complete extent or degree of an action, characteristic, property, state, structure, item, or result to function as indicated. For example, an object that is “substantially” enclosed would mean that the object is either completely enclosed or nearly completely enclosed. The exact allowable degree of deviation from absolute completeness may in some cases depend on the specific context, such that enclosing nearly all of the length of a lumen would be substantially enclosed, even if the distal end of the structure enclosing the lumen had a slit or channel formed along a portion thereof. The use of “substantially” is equally applicable when used in a negative connotation to refer to the complete or near complete lack of an action, characteristic, property, state, structure, item, or result. For example, structure which is “substantially free of” a bottom would either completely lack a bottom or so nearly completely lack a bottom that the effect would be effectively the same as if it lacked a bottom.

As used herein, the term “about” is used to provide flexibility to a numerical range endpoint by providing that a given value may be “a little above” or “a little below” the endpoint while still accomplishing the function associated with the range.

As used herein, a plurality of items, structural elements, compositional elements, and/or materials may be presented in a common list for convenience. However, these lists should be construed as though each member of the list is individually identified as a separate and unique member.

Concentrations, amounts, proportions and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 1 to about 5” should be interpreted to include not only the explicitly recited values of about 1 to about 5, but also include individual values and sub-ranges within the indicated range.

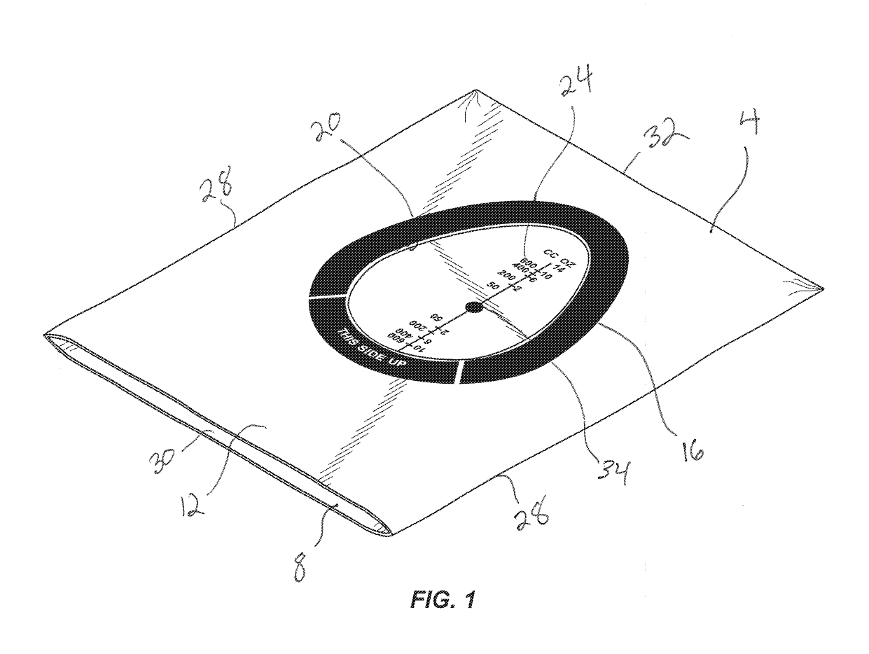

Turning now to FIGS. 1-7 there is shown a first embodiment of a biological specimen collection apparatus (4), made in accordance with the principals of the present disclosure. The biological specimen collection apparatus (4) may be formed by a first, lower layer or sheet (8) and a second, upper layer or sheet (12). The layers or sheets will typically be made from a liquid impenetrable material, such as polyethylene and may have a thickness similar to that of a conventional garbage bag.

The second, upper layer (12) includes a marking (16) to assist in aligning a hole (20), defined by the material of the upper layer, with the hole in the toilet seat. The marking (16) may also assist a user in properly sitting on the second, upper sheet (12) during use.

The lower sheet (8) may include a plurality of volumetric markings (24). The volumetric markings or gradations may generally be in alignment with the hole (20) in the second, upper sheet and may include a center marking to assist the user in placing the biological specimen.

As a person deposits a biological specimen such as urine, stool, or vomit, the markings provide a rough approximation of the volume of the specimen being maintained. There are several medical situations in which it is desirable to determine the volume of the specimen during use. For example, it may be desirable to ensure that a patient is urinating a volume which approximates the fluid intake to ensure that the patient's kidneys are functioning properly. Likewise, the volume of vomit may be noted.

The sides (28) and a distal end (32) of the biological specimen collection apparatus (4) may be either fused together or the upper and lower layers may be formed from one piece of material. The proximal end (30), however, may be open between the first, lower layer and the second, upper layer to facilitate mounting of the apparatus on a toilet seat.

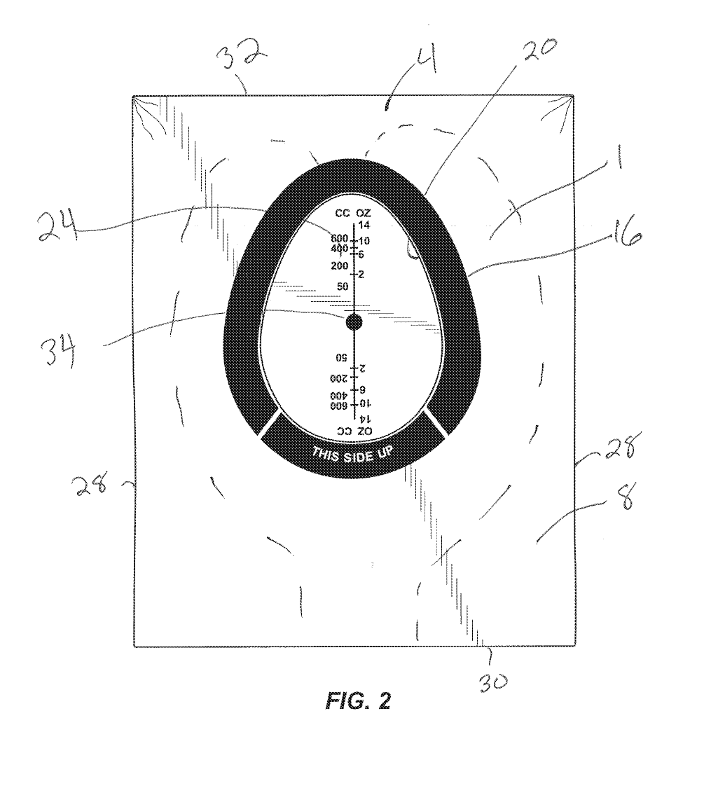

In use a toilet seat is raised slightly and the biological specimen collection apparatus (4) is slid onto the toilet seat by positioning the toilet seat between the first, lower layer (8) and the second upper layer (12) so that the toilet seat extends the through opening (30) so that the upper layer (12) is above the toilet seat and the lower layer (8) is below the toilet seat. The toilet seat (1) is shown in FIG. 2 by dashed lines.

Once the apparatus (4) is in place on the toilet seat (1), patient may deposit a biological specimen by sitting on the toilet or vomiting into the toilet, etc, wherein the specimen is received by the first, lower layer or sheet (8). Once the user has complete depositing the specimen the entire collection apparatus (4) may be removed from the toilet seat folded upwardly and tied so that the specimen is contained within the bag formed by the biological apparatus. A rubber band or other tie can be used to close the apparatus and keep the specimen inside until processed.

It would be appreciated that it would be most convenient to make the apparatus (4) from a light weight plastic material similar to that of a garbage bag—commonly polyethylene, though other materials may be used. The apparatus (4) may be made using a translucent, transparent, or opaque material depending on the desired uses.

FIG. 2 further shows how the marking (16) helps the user to properly position themselves as the toilet seat (1) would usually not be visible once the apparatus is mounted thereon. While a generally oval shape is shown in FIG. 2, other markings could also be used to designate proper positioning for the patient.

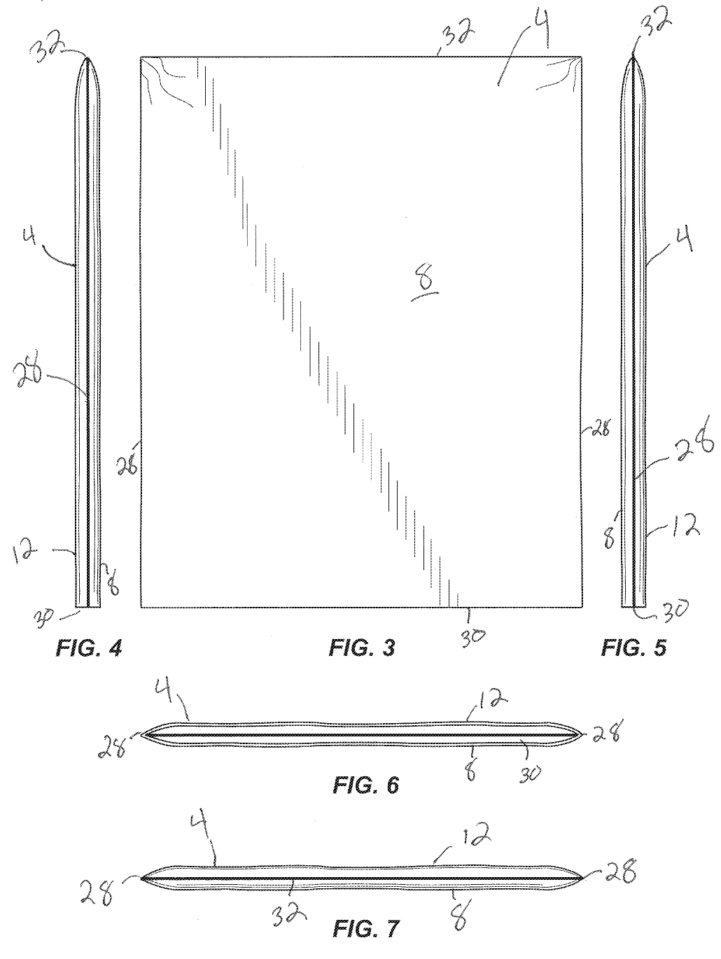

FIG. 3 shows the bottom of the apparatus so that only the first, lower layer (8) and the sides (28) are visible. The sides of the lower layer (8) may be creased or rounded.

FIGS. 4 and 5 show the sides (28) which may be formed as a continuous piece of material or may be formed by fusing the upper and lower layer together through a variety of well-known processes.

FIG. 6 shows an end view of the open end (30) of the apparatus (4) which enables the second, upper layer (12) and the first, lower layer (8) to be mounted on opposing sides of the toilet seat. The open end (30) will typically be wider than a conventional toilet seat to allow for easy installation.

FIG. 7 shows the distal, closed end (32) opposite the proximal, open end (30). The distal end 32 may either be formed by the first, lower layer (8) and the second, upper layer (12) being formed from an integral piece of material, or by bonding or fusing adjacent the seam.

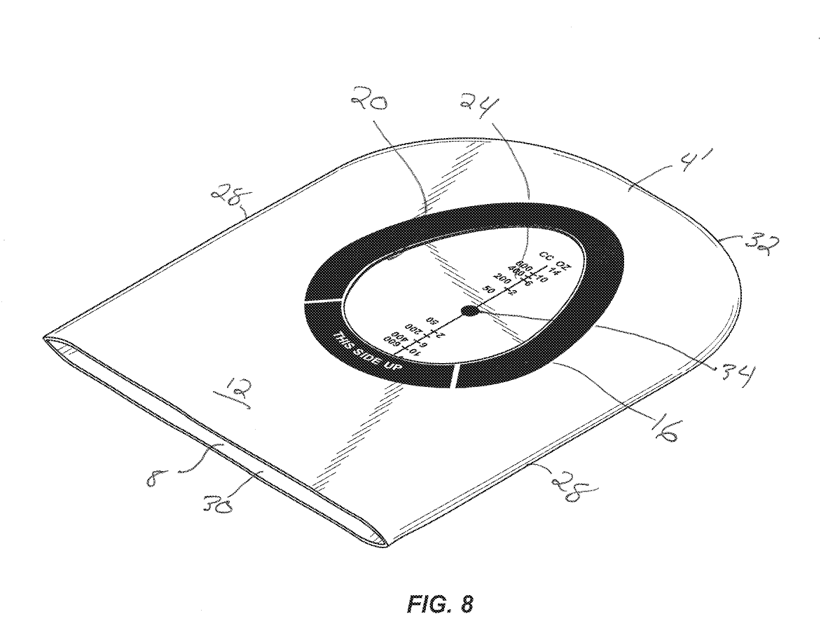

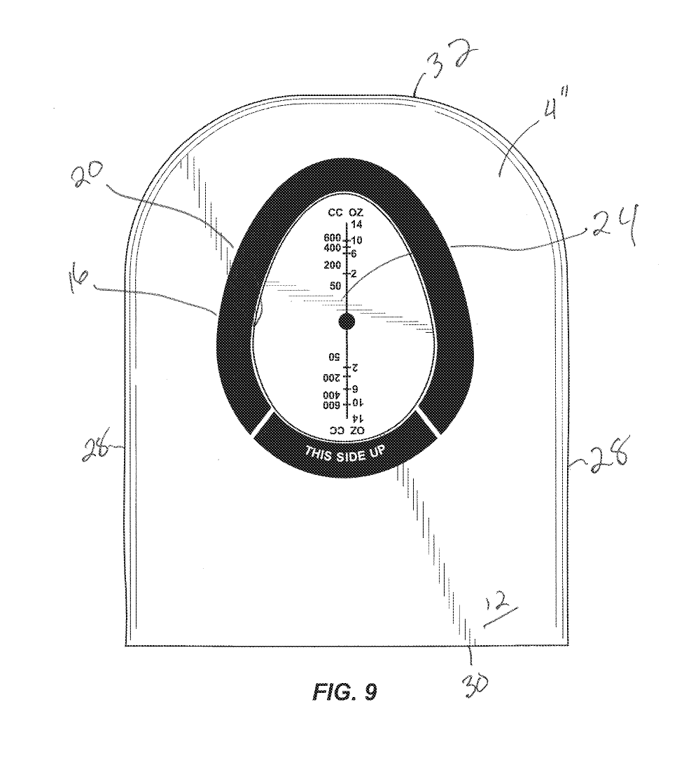

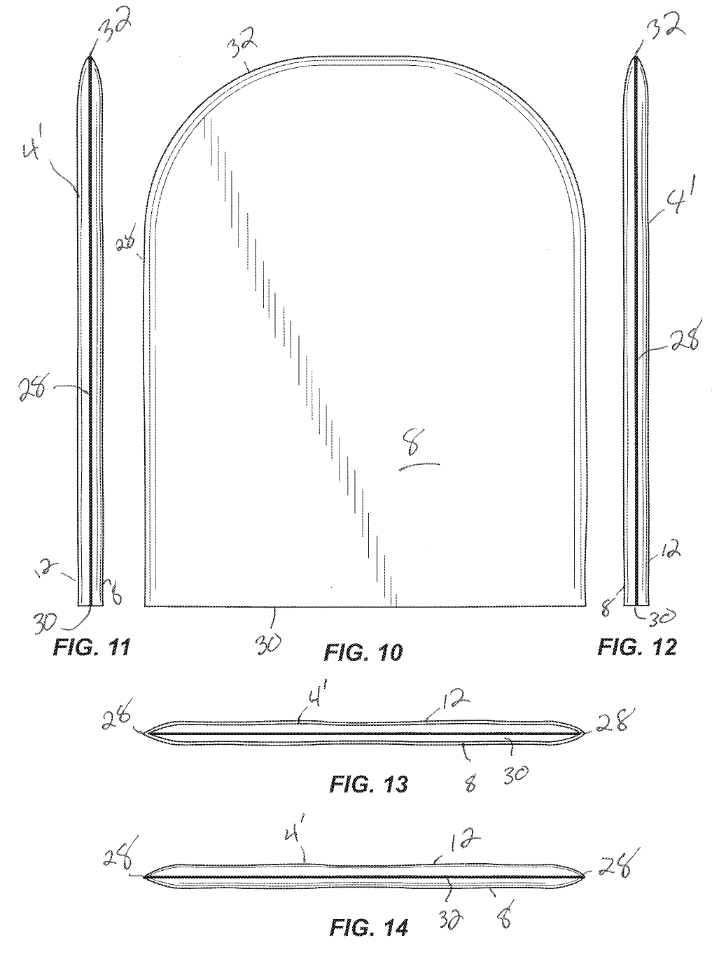

Turning now to FIG. 8, there is shown an alternate embodiment. The various aspects of the embodiment are generally the same except that the distal end (32′) is rounded, instead of being squared off like the embodiment shown in FIG. 1. Thus, while the biological specimen collection apparatus (4) of FIGS. 1-7 has a generally rectangular shape, the biological specimen collection apparatus (4′) of FIGS. 8-14 has the closed end (32) rounded so as to more closely approximate the shape of a toilet seat. Otherwise the apparatus (4′) is substantially the same as that discussed above with FIGS. 1-7 and FIGS. 8-14 are numbered accordingly. The discussion regarding FIGS. 1-7 is hereby incorporated by reference.

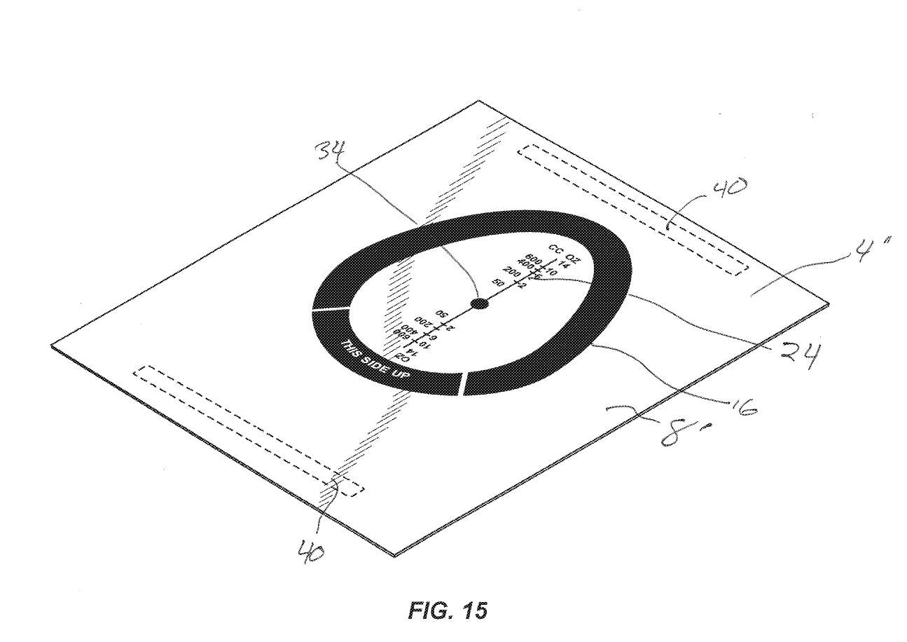

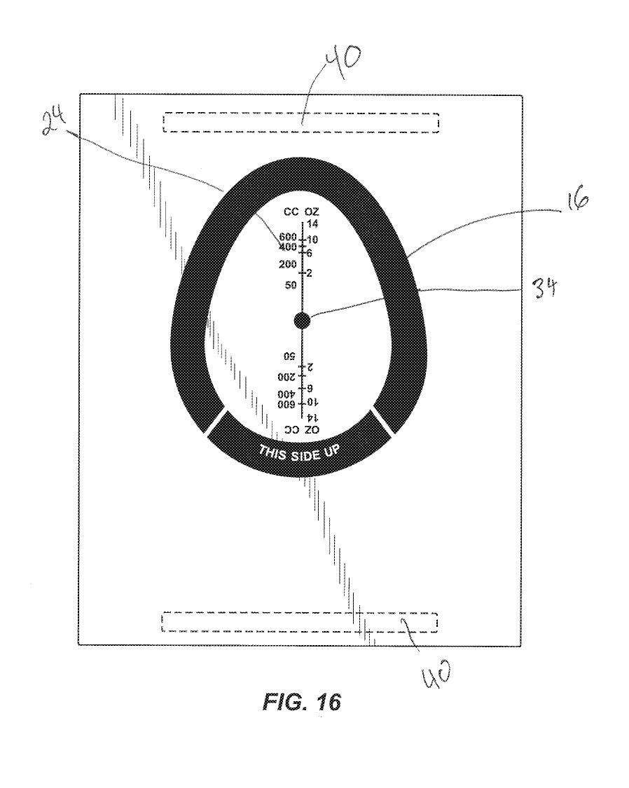

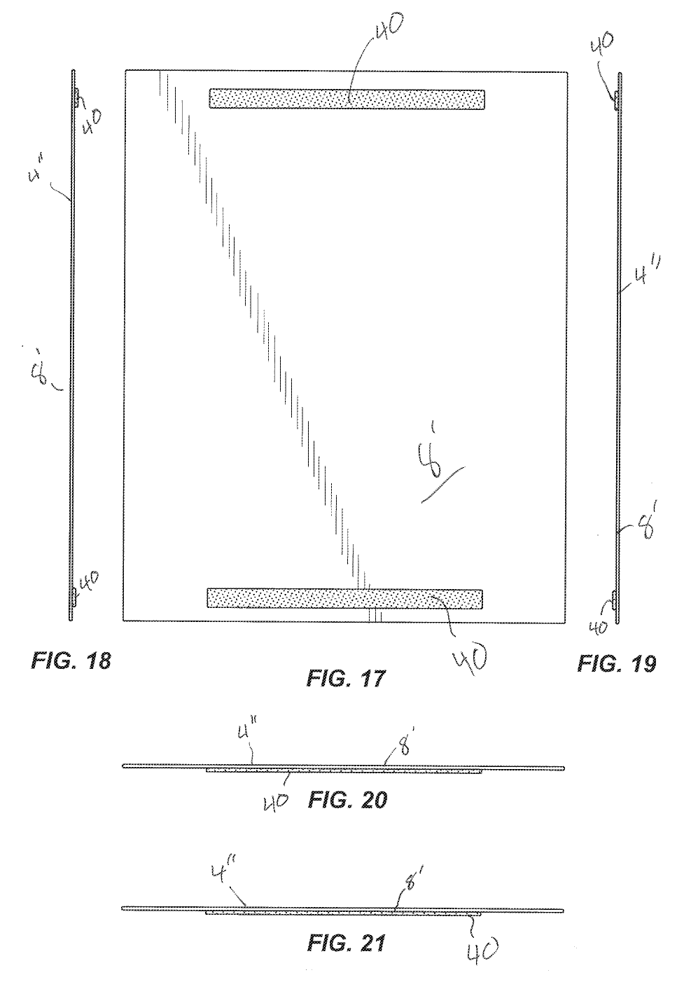

Turning now to FIG. 15, there is shown an alternate embodiment of a biological specimen collection apparatus (4″) in accordance with the present disclosure. Rather than using an upper layer and lower layer so as to encase the toilet seat during use, the apparatus (4″) shown in FIG. 15 shows a single sheet (8′). The sheet (8′) may include markings (16) to facilitate alignment with the hole in the toilet seat (not shown) and a plurality of volumetric markings (24) or gradations to give an approximation of volume received by the biological specimen collection apparatus (4″). Likewise, it may include a center marking (34) to assist in volumetric determination/aiming.

Rather than being disposed on the top and bottom of the toilet seat (1, FIG. 2), the sheet (8′) may include a plurality of adhesive attachments members (40). The adhesive attachment members (40) are used to attach the sheet to the toilet bowl so that the sheet will remain stable in use.

Thus, the toilet seat is lifted and the adhesive attachment members (40) are exposed. This will typically be done by pulling off one or more cover strip or other protective layer to expose the adhesive, which is typically on the underside of the sheet (8′). The adhesive is then applied to the toilet bowl to hold the sheet (8′) in place during use. While two adhesive attachment members (40) are shown, one large adhesive attachment member or a number of smaller ones may be used.

Preferably the person attaching the sheet (8′) positions the sheet so that the marking (16) is in alignment with the opening in the toilet seat. The toilet seat is then returned to its position so that the patient may urinate, defecate, or vomit into the area provided with the volumetric markings (24). Once the patient has used the device medical personnel can come and make a notation as to the approximate volume and remove the sheet from the toilet and tie it off so that the biological specimen can be analyzed.

While shown is generally a rectangular sheet it will be appreciated that sheet (8′) could be rounded on the end or otherwise configured to engage a toilet seat. It will also be appreciated that the sheet (8′) may be attached to the toilet bowl at the front and rear. The sheet (8′) may include additional adhesive strips to allow attachment to the sides as well. It will be appreciated that the medical personnel may not want to pull the sheet total (8′) taut so that sheet can sag and indicate the approximate volume of the received specimen.

Thus, there is disclosed a biological specimen collection apparatus. It will be appreciated that in light of the present disclosure, numerous modifications can be made without departing from the scope and spirit of the invention. The appended claims are intended to cover such modifications.

What is claimed is:

1. A biological specimen collection apparatus comprising at least one sheet of material configured for disposal above a toilet bowl so as to collect a biological specimen and a marking disposed on at least one sheet of material for aligning the material relative to an opening in a toilet seat.

2. The apparatus of claim 1, wherein at least one sheet of material comprises a plurality of volumetric markings.

3. The apparatus of claim 1 where in at least one sheet of material comprises a first lower layer and second upper layer having an opening at one end configured to enable the toilet seat to be slid between the lower layer and the upper layer.

4. The apparatus of claim 1, wherein the marking is generally ovular in shape.

5. The apparatus of claim 4, wherein the at least one sheet of material comprises a first lower layer and a second, upper layer, the second upper layer having a hole inside of the marking.

6. A system for capturing biological fluid, the system comprising:
an upper layer having sides, a distal end, and a proximal end, and defining a hole therethrough; and
a lower layer having sides, a distal end, and a proximal end;
wherein the sides and distal end of the upper layer are connected to the sides and distal end of the lower layer, and the proximal end is generally open.

7. The system of claim 6, wherein the upper layer and lower layer are formed from a single piece of material.

8. The system of claim 6, wherein the proximal end is configured to be mounted on a toilet seat.

9. The system of claim 6, wherein the lower layer comprises a plurality of volumetric markings.

10. The system of claim 9, wherein the upper layer comprises one or more markings to assist aligning the hole therethrough with a hole of a toilet seat.

11. The system of claim 10, wherein the upper layer comprises one or more markings to assist a user to properly sit on the upper layer.

12. The system of claim 11, wherein the lower layer comprises a continuous sheet of material configured to capture liquids and solids.

13. The system of claim 9, wherein the distal ends are substantially semicircular.

14. The system of claim 9, wherein the hole therethrough is a generally round hole formed towards the distal end of the upper layer.

15. The system of claim 6, wherein the upper layer and lower layer are fused together on their sides and distal ends.

16. A system for capturing biological fluid, the system comprising:
a layer of material having sides, a distal end, and a proximal end, the layer comprising a plurality of volumetric markings;
an adhesive attachment member configured to attach the layer of material to a toilet bowl; and
wherein the layer of material is substantially continuous and configured to capture liquids and solids.

17. The system of claim 16, wherein the adhesive attachment member comprises two or more adhesive attachment members.

18. The system of claim 16, wherein the layer of material comprises one or more markings to assist aligning the layer of material with a toilet bowl.

19. The system of claim 12, wherein the upper layer and lower layer are formed from polyethylene.

20. The system of claim 12, wherein the lower layer is configured to be closed around a sample.
